(19) United States
(12) Patent Application Publication
(10) Pub. No.: US 2025/0160962 A1
(43) Pub. Date: May 22, 2025

(54) SHARED SET OF OBJECT REGISTRATIONS FOR SURGICAL DEVICES USING INDEPENDENT REFERENCE PLANES

(71) Applicant: Cilag GmbH International, Zug (CH)

(72) Inventors: Frederick E. Shelton, IV, Hillsboro, OH (US); Jacqueline Corrigan Aronhalt, Loveland, OH (US); Jason L. Harris, Lebanon, OH (US)

(21) Appl. No.: 18/810,230

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. A61B 34/20; A61B 1/00; A61B 1/313; A61B 90/00; G16H 30/40

(52) U.S. Cl. CPC A61B 34/20; A61B 1/00009; A61B 1/0005; G16H 30/40; A61B 1/3132; A61B 2034/2065; A61B 2090/363; A61B 2090/365; A61B 2090/373

(57) ABSTRACT

Devices, systems, and techniques for a shared set of object registrations for devices with independent reference planes. An example imaging device may capture a first imaging data stream of a visualized surgical structure. The imaging device may communicate registration information with another surgical imaging device capturing a second imaging data stream of the visualized surgical structure. The imaging device may identify, based on the registration information, a first location, within the first imaging data stream, associated with the visualized surgical structure. The first location may be determinable based on: a first reference geometry associated with a field of view of the first surgical imaging device, a second reference geometry associated with a field of view of the second surgical imaging device, a transformation between the first reference geometry and the second reference geometry, and a reference landmark common to the first imaging data stream and the second imaging data stream.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023,
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

This application is related to the following, filed contemporaneously, the contents of each of which are incorporated by reference herein:
U.S. patent application Ser. No. 18/810,208, filed Aug. 20, 2024.

### BACKGROUND

With the complexity and autonomy of smart devices, particularly in the medical field, interactions may be managed between multiple smart devices (e.g., and legacy devices). Systems may operate in isolation or with limited collaboration, limiting their effectiveness and potentially leading to instability or predictability failures. Means for coordinating these systems may be static and may not adapt based on changing circumstances or patient parameters, posing a potential challenge in providing patient care and monitoring.

### SUMMARY

Within healthcare, systems may facilitate an environment conducive to medical practices. A first system may interact and/or coordinate with one or more other system(s). Shared object registration may enable the systems to identify common objects in the systems' respective fields of view. One system may register surgical structures in its field of view and share the registration information with another system.

Systems utilizing shared object registration may have different reference planes (e.g., independent local reference planes). For example, the systems may include respective surgical scopes that are on opposite sides of a tissue barrier (e.g., an organ wall). As a result, the first system may view objects in different locations and/or at different angles than the second system. To enable the systems to accurately use the shared object registrations, the independent local reference planes may need to be aligned with each other.

A first system may have pre-operative imaging and a second system may have intra-operative imaging. The patient may be in a different position in the pre-operative imaging compared to the intra-operative imaging. The change in patient position may cause surgical structures (e.g., organs, tumors, etc.) to shift, thereby exacerbating the differences between the two imaging systems. The shared object registration may enable the systems to use non-moving or less affected objects as baseline landmarks for aligning other surgical structures.

### BRIEF DESCRIPTION OF THE DRAWINGS

Examples described herein may include a Brief Description of the Drawings.

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 is a data flow diagram illustrating communication between an endoscopic scope and a laparoscopic scope.

FIG. 6 illustrates example components of a landmark identification and matching engine.

FIG. 7 illustrates an example method for object registration.

FIG. 8 illustrates an example of two scopes registering objects in their field of view.

FIG. 9A illustrates example organ positions while a patient is in the supine position.

FIG. 9B illustrates example organ positions while the patient is in the Trendelenburg position.

FIG. 10A illustrates example gallbladder anatomy while a patient is in the supine position.

FIG. 10B illustrates example gallbladder anatomy while the patient is in the Trendelenburg position.

FIG. 11 illustrates an example comparison of gallbladder anatomy in pre-operative imaging and gallbladder anatomy in intra-operative imaging.

FIG. 12A illustrates example gallstone positions while a patient is in the supine position.

FIG. 12B illustrates example gallstone while the patient is in the Trendelenburg position.

FIG. 12C illustrates an example of adjusting an imaging location based on a change in patient position.

FIG. 13 illustrates an example method that may be performed by a surgical instrument.

### DETAILED DESCRIPTION

A more detailed understanding may be had from the following description, given by way of example in conjunction with the accompanying drawings.

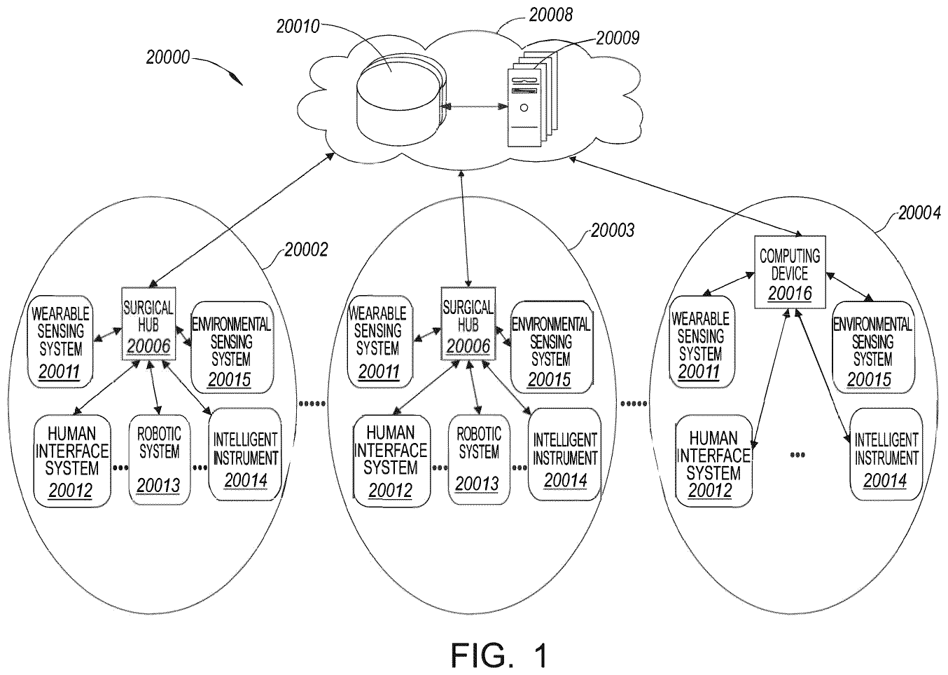

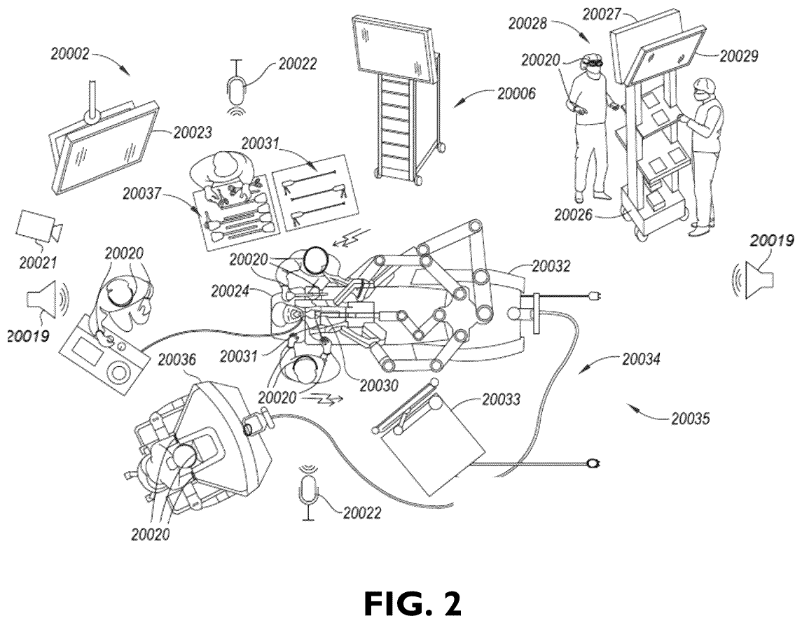

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LoWPAN), Wi-Fi.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

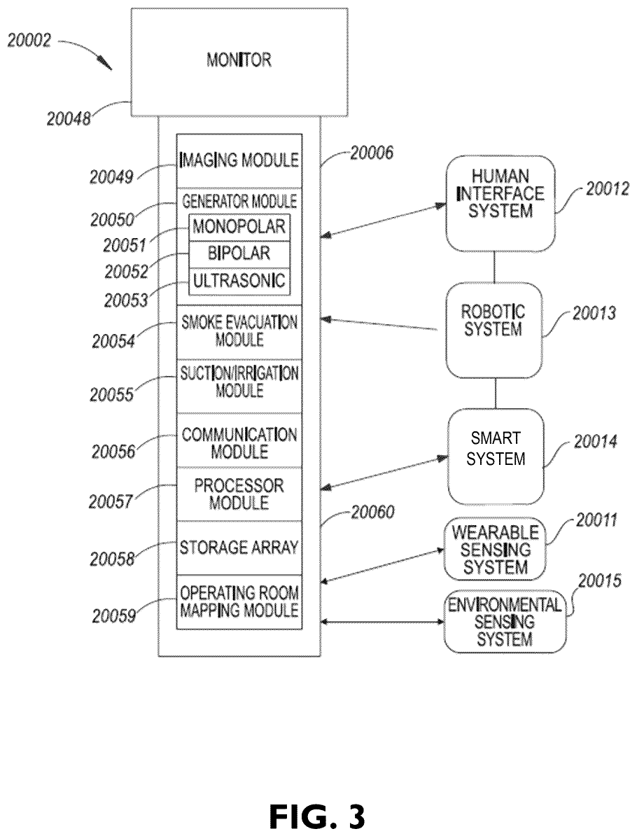

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces off the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

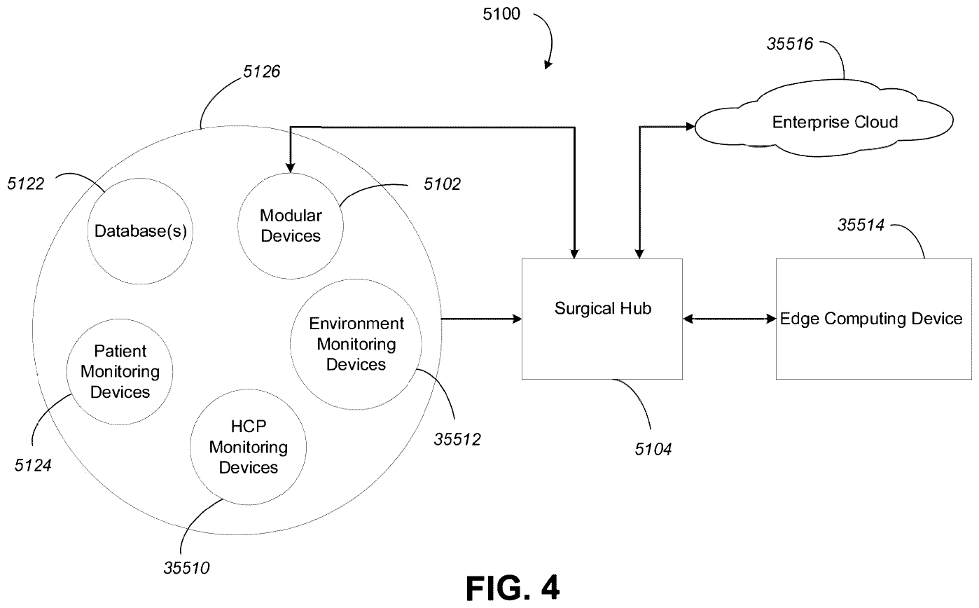

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient monitoring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

Within healthcare, systems may facilitate an environment conducive to medical practices. A first system may interact and/or coordinate with one or more other system(s). Shared object registration may enable the systems to identify common objects in the systems' respective fields of view. One system may register surgical structures in its field of view and share the registration information with another system.

Systems utilizing shared object registration may have different reference planes (e.g., independent local reference planes). For example, the systems may include respective surgical scopes that are on opposite sides of a tissue barrier (e.g., an organ wall). As a result, the first system may view objects in different locations and/or at different angles than the second system. To enable the systems to accurately use the shared object registrations, the independent local reference planes may need to be aligned with each other.

A shared set of object registrations between two independent reference planes (e.g., from separate imaging streams) may be used to allow systems to locate common registered objects (e.g., in one or more of the independent local reference planes). For example, a first surgical imaging device may capture a first imaging data stream of a visualized surgical structure. The first surgical imaging device may communicate registration information with a second surgical imaging device that is capturing a second imaging data stream of the visualized surgical structure. The first surgical imaging device and the second surgical imaging device may be capturing video data at the same time. The first surgical imaging device may identify (e.g., based on the registration information) a first location (e.g., within the first imaging data stream) associated with the visualized surgical structure. The first location may be determinable based on: a first reference geometry associated with a field of view of the first surgical imaging device, a second reference geometry associated with a field of view of the second surgical imaging device, a transformation between the first reference geometry and the second reference geometry, and a reference landmark common to the first imaging data stream and the second imaging data stream.

The first surgical imaging device may calculate the transformation between the first reference geometry and the second reference geometry based on a first physical orientation of the first surgical imaging device, a second physical orientation of the second surgical imaging device, and the reference landmark.

A system may use the shared registrations (e.g., associated with operational usage originating from a second independent system) for common use. A first system may determine the registration of visualized surgical structures. A second (e.g., separate) system may share handling and usage of the registration. The shared registration may be between two independent visualization systems. The shared usage of the registration may result in navigation of a moving system using the registration of locations generated by an independent system (e.g., not coupled to the motions of the moving system). The shared registration may have a deterministic aspect that enables the combined overall system to differentiate between objects. In some examples, the visualized surgical structure may be an object to be removed from a patient, and the reference landmark may be an anatomical structure of the patient.

Multiple systems may perform object imaging synchronization. The systems may perform deterministic identification of interchangeable registrations. The systems may perform pre-operative imaging adjustment for real-time use. A real-time user may use interchangeable registrations of multiple visualization sources.

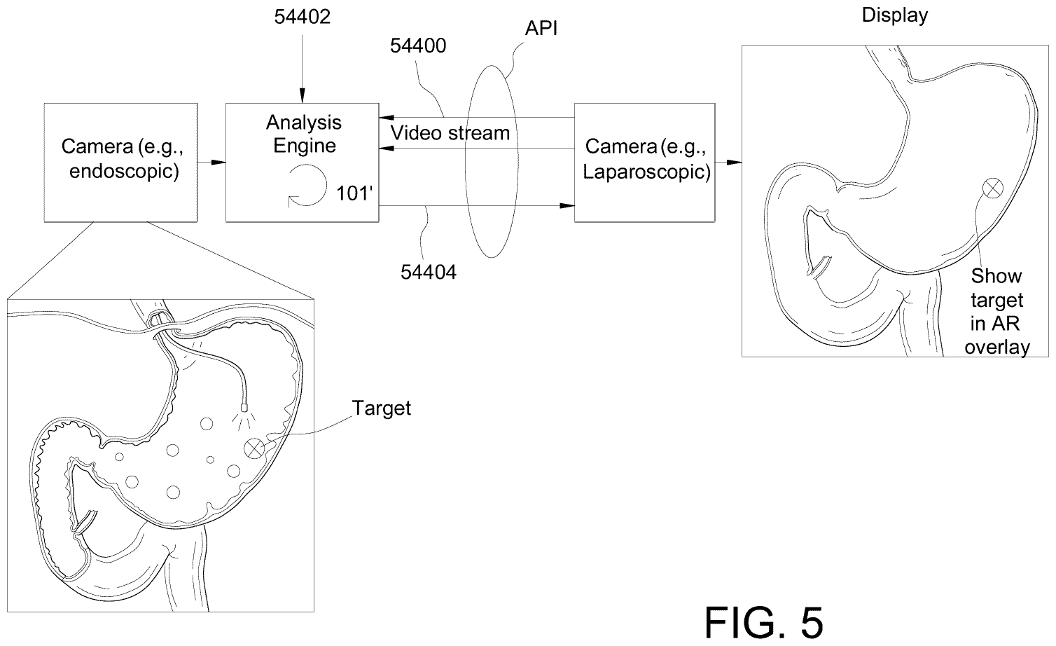

If multiple systems capable of visualization are connected, the visualization data from the systems may be combined (e.g., within the primary system's processor), as illustrated in FIG. 5. A display may be used to merge the visualization sources from the systems. The merged visualization data may be displayed to the user. Common registration points may be used as the foundation of the merged the visualization data.

The first surgical imaging device may be an endoscopic device, and the second surgical imaging device may be a laparoscopic device (e.g., the first surgical imaging device and the second surgical imaging device may be separated by a tissue barrier). For example, as illustrated in FIG. 5, an endoscopic camera may identify a target object. The endoscopic camera may send the location information associated with the target object to an analysis engine. The analysis engine may receive another input 54402 (e.g., a global reference geometry, a transform equation, and/or the like). The analysis engine may adjust the location information based on the other input 54402 and output the adjusted location information 54404 to a laparoscopic camera (e.g., the analysis engine and laparoscopic camera may communicate via an API). The laparoscopic camera view may be from the opposite side of a tissue barrier as the endoscopic camera (e.g., inside and outside the stomach, as shown).

The laparoscopic camera may share location information associated with the orientation of the laparoscopic camera. For example, the laparoscopic camera may send its reference plane geometry to the analysis engine. The analysis engine may use the location information from the endoscopic camera and the laparoscopic camera to generate a transform between the reference geometries of the cameras. The analysis engine may reanalyze the location of the target object based on the generated transform to more accurately indicate where the target object is in the laparoscopic camera's view. The laparoscopic camera may send a live feed of its view to a display.

One of the smart surgical devices (e.g., cameras) may generate an augmented reality (AR) overlay of the first imaging data stream, the second imaging data stream, based on the transformation between the first reference geometry and the second reference geometry. The AR overlay may depict the first imaging data stream and the second imaging data stream overlapped onto one another based on the transformation. The AR overlay may include an indication of the location of the target object (e.g., determined based on the transform information from the analysis engine). The smart surgical device may output the AR overlay to a display device for displaying. The display may include the AR overlay.

Imaging systems (e.g., two separate imaging systems) may monitor a common surgical site from different points-of-view (e.g., possibly on differing side of a common organ wall). The views from the imaging systems may be switched and/or overlayed. Multiple imaging sources may be integrated. For example, the views may be overlaid on one another. The imaging systems may provide a switchable point of view (POV). Occluded viewing angles may be reduced. Organ(s) and/or tissue may be in the way (e.g., blocking the user's view). For example, a surgeon may not be able to see an instrument from a desired angle. For example, the surgeon may not be able to monitor an instrument in real time (e.g., using externally visible motion). The surgeon may want to monitor tissue positioning within jaws of an instrument. For example, the surgeon may need to be aware of an incompatible bite or tissue that is not fully captured within the active portion of the jaws. A user may desire to switch between imaging systems (e.g., two separate imaging systems having different approach angles). For example, the different approach angles may be from an endoscopic device and a laparoscopic device.

A plurality of systems may be integrated into a central system (e.g., a hub). Example communication layers are provided herein. A communication layer of a video signal may be used (e.g., by itself). Other information may be included in the video signal (e.g., an ML model to arrive at orientation, and/or the like). The communication layer of the video signal may be stamped or coupled with additional information (e.g., digitally). For example, the information may be timestamp information, camera orientation information, and/or the like. The information may aid in the synchronization of the systems.

A multi-camera (e.g., two-camera) procedure may be performed. For example, a first vision system may be located within an organ, and a second vision system may be located exterior to the organ. In a colorectal procedure, for example, a smart visualization system may be utilized for the patient. The visualization system may include multiple (e.g., two) coordinating camera systems. A first camera system may be inserted into the organ (e.g., to provide internal visualization), and a second camera system may be used to showcase a targeted area for a surgical instrument (e.g., a stapler).

In an example, a laparoscopic instrument may approach a tumor from the outside wall of the colon. A colonoscope within the colon may have robotic control of the local insertion, and/or movement of the camera (e.g., up, down, left, right, and rotational control). The surgeon may know where the endoscopic instrumentation (e.g., and associated view) is with respect to the laparoscopic instrument(s) outside the colon. The laparoscopic instruments may assist the access and resection procedures (e.g., that are taking place within the colon). Other hollow organs, such as the bladder, may use similar techniques as that described herein with respect to the colon.

An endoscopic device may calculate the transformation between the first reference geometry and the second reference geometry based on one or more lights projected, by the laparoscopic device, through the tissue barrier, and detected by the endoscopic device.

For example, a laparoscopic device with a light source (e.g., a sub-millimeter LED on laparoscopic tool) may be in contact with the colon. The light source may allow the endoscopic view to determine the position of the laparoscopic device. The endoscopic device may adjust its movements to ensure the endoscopic device and related instrumentation is driven to the location of the laparoscopic device (e.g., the cooperative site). In an example, a laparoscopic device may pinch and stretch an area around a tumor. The location of the laparoscopic device may be pinpointed using the light source. The endoscopic device may be driven to the light source (e.g., LED pinpoint) if the endoscopic device sees the light source on the endoscopic side. Once the endoscopic device reaches the cooperative site, working instruments such as graspers, injectors, cutters, and/or the like may be introduced to that site. Light (e.g., from an LED) may be projected as a laser dot from a laparoscopic shaft. The LED may be in a tethered or untethered capsule. The LED may be dropped into the laparoscopic field of view. The laparoscopic device (e.g., a laparoscopic grasper) may pick up the LED and (e.g., manually) position it as needed. In this case, the endoscopic device may use the LED pinpoint orientation as a guiding point (e.g., North Star).

A fluorescing system may be used to identify the inferior mesenteric artery (IMA) and/or adjunct vascular structures. The IMA (as seen from a first imaging system) may be used to generate an augmented view of another system (e.g., a laparoscopic view and/or endoscopic view). The IMA may be detected with a first imaging system through NIR with ICG. The imaging system may combine that position of IMA with an endoscopic and/or laparoscopic view.

The flexible endoscopy robot and the laparoscopic robot may be controlled by separate arms of the same Hugo robot. The Hugo visualization system may be coupled to the laparoscopic robot site. The flexible endoscopic scope may be an autonomous visualization unit or a second coupled unit within the same Hugo system. In this case, the use of IMA fluorescing may differ. If multiple independent systems are sharing registrations, critical structures may be used as the initial pathway to communicate imaging data bi-directionally.

A smart laparoscopic robot and visualization may couple to a hand-held flexible endoscope with an autonomous endoscope visualization source. If a smart system (e.g., that is capable of visualization) is connected to a second smart system (e.g., that is also capable of visualization), the systems may pre-identify themselves as primary or secondary. If a system requests capacity for an action, the visualization systems may alternate as needed to perform the action (e.g., based on the data capacity of the systems). If the primary systems visibility becomes obscured or limited, the visualization may switch to the secondary system. The secondary system may yield visualization to the primary system (e.g., as long as the primary systems visibility is within an operational envelope).

If the visualization system of a first device (e.g., a smart laparoscopic robot) is superior to that of a second device connected to the first device, the smarter system may prioritize its own visualization source to be displayed (e.g., and ignore the secondary, inferior source). The superior smart visualization source may communicate to the secondary source to stop transmitting visualization data (e.g., because it won't be used). The superior smart visualization source may mirror back the visualization data from the connected secondary source (e.g., so that the secondary source believes it is operating as normal and will not cause error codes). If the superior smart visualization source is not displaying data from the secondary connected source, the superior source may receive the secondary data as backup (e.g., if needed). The secondary data (e.g., second feed) may be used to confirm registration and/or the accuracy of the display. The secondary data may be used (e.g., if needed) to extend the visualization display area.

If multiple visualization systems are connected, a visual indicator (e.g., LED on device, vision system source display on screen, etc.) may be displayed to let the user know the source of the visual feed (e.g., if the vision feed is coming from an autonomous endoscope source or from a smart laparoscopic robot). The system that is being used to provide the visual feed may be based on the procedure or user preference.

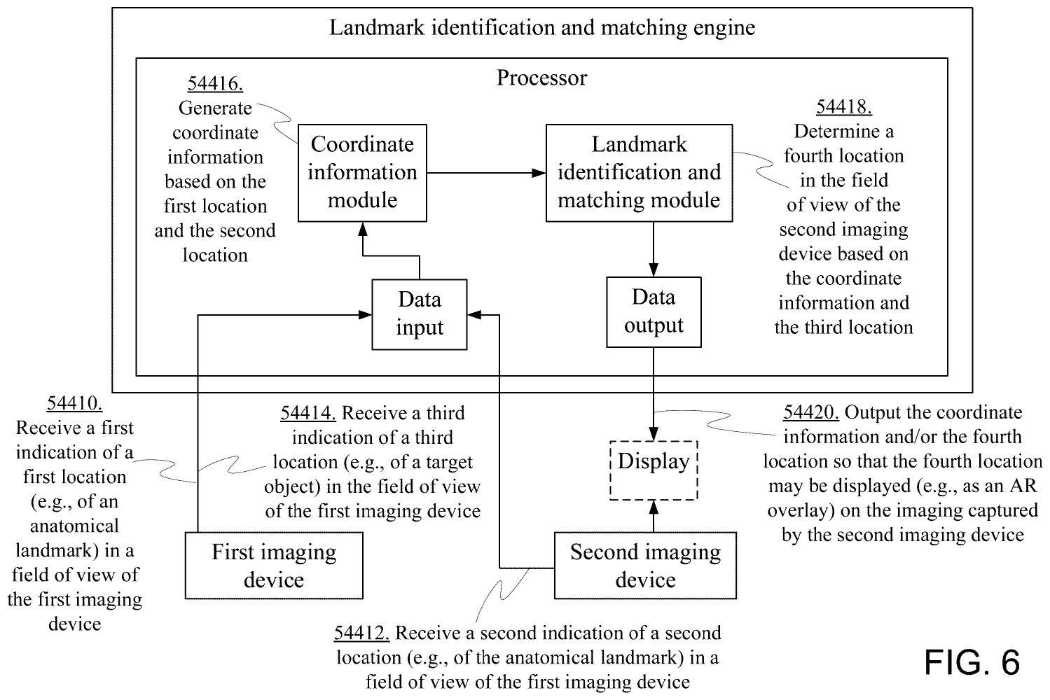

FIG. 6 illustrates example components of a landmark identification and matching (LIM) engine (e.g., such as the analysis engine in FIG. 5). As shown, the LIM engine may include a processor with one or more modules. The LIM engine may be in communication with a first and second imaging device. One or more of the imaging devices and/or the LIM engine may be connected to a display.

As illustrated, at 54410, the LIM engine may receive (e.g., via a data input module) a first indication of a first location in the field of view of the first imaging device. The first location may be indicative of the location of an anatomical landmark (e.g., a tumor, major organ structure, etc.). At 54412, the LIM engine may receive a second indication of a second location in the field of view of the second imaging device. The second location may be indicative of the location of the same anatomical landmark (e.g., from a different angle and/or side of a tissue barrier).

At 54414, the LIM engine may receive a third indication of a third location in the field of view of the first imaging device. The third location may be indicative of the location of a target object (e.g., tumor, incision, etc.). The LIM engine may, at 54416, use a coordinate information module to generate coordinate information based on the first location and the second location. At 54418, the LIM engine may use a landmark identification and matching module to determine a fourth location in the field of view of the second imaging device that corresponds to the location of the target object. The LIM engine may determine the fourth location based on the coordinate information and the third location.

The second imaging device may display a live feed of its field of view on a display for a user. The LIM engine may, at 54420, output the coordinate information and/or the fourth location so that the fourth location can be displayed on the display. For example, the fourth location or the coordinate information may be added as an AR overlay on the imaging captured by the second imaging device. The overlay may allow a user (e.g., surgeon) to locate the target object without having a direct visual of the target object.

A secondary device may be integrated into a primary device. A handheld device may be integrated into a robotic device (e.g., where the robotic device is the central device). A robotic device may be integrated into a handheld device (e.g., where the handheld device is the central device).

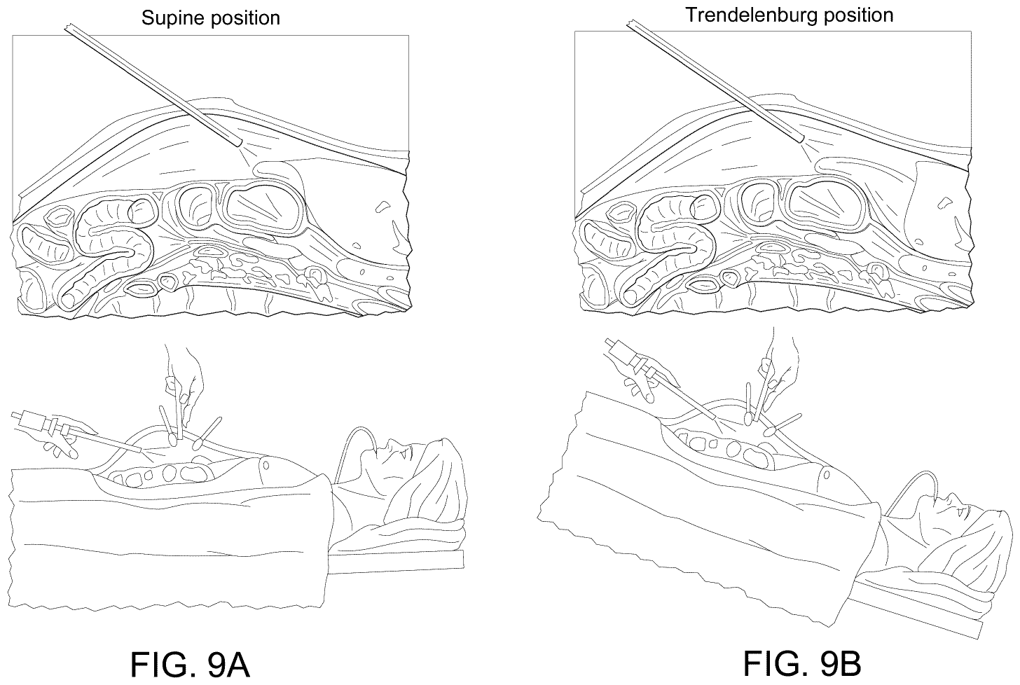

A patient may be positioned differently for pre-operative imaging and intra-operative imaging. For example, one position may provide better visibility of internal structures being imaged pre-operatively, and another position may provide the surgeon better access to those structures during the operation, as illustrated in FIGS. 9A and 9B. Some example patient positions are described herein.

Supine position refers to a patient laying on their back horizontally. Supine position (e.g., horizontal supine position) may be used for head, face, chest, abdomen, and/or limb (e.g., lower limb) surgery. The supine position may be used in abdominal surgery, gynecological surgery, and/or orthopedic surgery.

Oblique supine position (e.g., patient tilted to one side by 45 degrees) may be used for anterior lateral approach, lateral chest wall, axillary surgery, etc. Side head supine position may be used for ear, maxillofacial, side neck, head surgery, and/or the like. Upper limb abduction supine position may be used for upper limb and breast surgery.

Lateral position (e.g., patient laying on their side) may be used for chest incision surgery, hip surgery, and/or the like. The lateral position may be used for neurosurgery, thoracolumbar surgery, hip surgery, and/or the like. The lateral position may provide sufficient exposure of the surgical field for convenient operation by the surgeon. The lateral position may cause changes in the patient's physiology (e.g., which may lead to complications such as circulation, breathing disorders, nerve damage, and/or skin bedsores).

General surgery chest lateral position may be used for lung, esophagus, side chest wall, and/or side waist (e.g., kidney and ureter middle and upper part) surgery, etc. Lateral position may be used for kidney and ureter middle and upper section surgery. The distance between the lower rib to the lumbar bridge may be three centimeters (e.g., which may be suitable for kidney surgery, nephrectomy, ureteral stone removal). If a lumbar bridge is not present, the “folding bed” may be in a flex position. Lateral position may be used for hip surgery (e.g., for acetabular fracture combined with posterior dislocation of the hip, artificial hip replacement, quadratus femoris bone flap transposition for the treatment of aseptic necrosis of the femoral head, open reduction and internal fixation of femoral shaft fractures, femoral tumors, femoral neck fractures or intertrochanteric fractures, internal fixation and upper femoral osteosynthesis, etc.).

The Trendelenburg position (e.g., supine position with head down) is a variation of the supine position. Trendelenburg position may be used in head, neck, thyroid, anterior cervical surgery, cleft palate repair, general anesthesia, tonsillectomy, tracheal foreign body, esophageal foreign body surgery, and/or the like. The Trendelenburg position may be accompanied by a small downward fold of the leg plate. The Trendelenburg position may be used for laparoscopic surgery, or pelvic or lower abdomen surgery. The patient's posture may be able to move to the supine position (e.g., under normal conditions or in emergency situations, for example, if the power supply is interrupted). FIGS. 9A and 9B illustrate how the Trendelenburg position may provide a surgeon with better access to some organs compared to the supine position.

Reverse-Trendelenburg (e.g., supine position with head up) is a variation of the supine position with a forward tilt. Reverse-Trendelenburg position may be used for head and neck surgery, and/or abdominal procedures (e.g., bariatrics). For example, the Reverse-Trendelenburg position may be used in head and neck surgery to reduce venous congestion and prevent gastric reflux (e.g., during the induction of anesthesia). In abdominal procedures, Reverse-Trendelenburg position may be used to allow gravity to pull the intestines lower (e.g., providing easier access to the stomach and adjacent organs).

In the Jack-knife position (e.g., sometimes referred to as the Kraske position) a patient's head and feet are lower than the patient's hips. Jack-knife position may be used for gluteal muscle and anal (e.g., rectal) surgery. In gallbladder and kidney surgery (e.g., in the absence of a lumbar bridge), the back and buttocks may be folded to form an arch (e.g., to replace the lumbar bridge). The arched starting point may be fixed. The height may be limited by the folding angle.

The patient surgical position may impact the insufflation pressure, the peripheral blood flow, the anesthesia magnitude, breathing, heart rate, and/or the like. While the patient surgical position may provide better access to the surgical site, the position may affect related system performance and thresholds (e.g., smart digital system closed loop performance and thresholds). For example, compared with the mean inflated volume for the supine position (e.g., 3.22±0.78), the mean inflated volume may increase by 900 ml for the Trendelenburg position or if the legs are flexed at the hips, and may decrease by 230 ml for the reverse-Trendelenburg position.

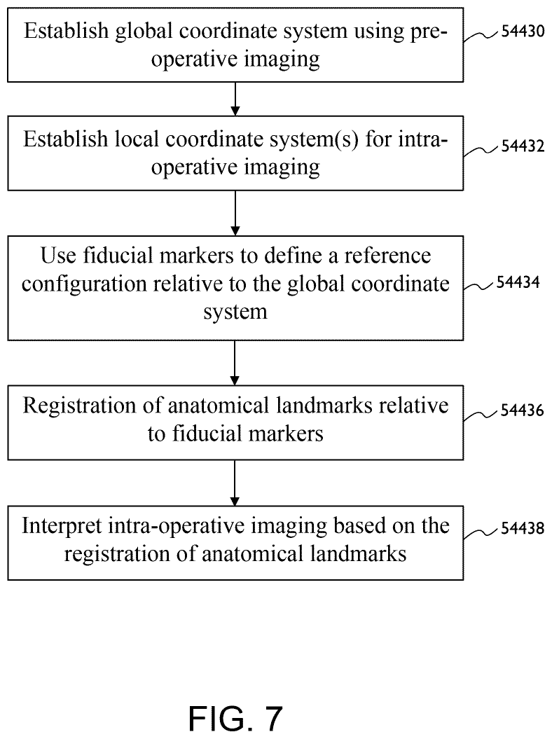

FIG. 7 illustrates an example method for object registration. At 54430, a system may establish a global coordinate system (e.g., using pre-operative imaging). The system may, at 54432, establish one or more local coordinate system(s) for intra-operative imaging. As discussed herein, the patient's position may have changed between the pre-operative imaging and the intra-operative imaging. Accordingly, anatomical structures may have shifted/changed locations.

At 54434, the system may use fiducial markers to define a reference configuration relative to the global coordinate system. The system may, at 54436, perform object registration of anatomical landmarks relative to the fiducial markers. At 54438, the system may interpret the intra-operative imaging based on the registration of the anatomical landmarks. For example, the system may identify the anatomical landmarks even though they have shifted since the pre-operative imaging was performed.

In examples (e.g., if multiple imaging platforms/technologies are in play), communication between the systems may help with coordination techniques described herein. The endoscopic view (e.g., flexible endoscopic view) may have visualization. The endoscopic view may have ultrasound imaging onboard. The laparoscopic camera may have (e.g., only have) the visual image. Connecting the information from the two systems and the pre-operative imaging (e.g., which may provide more or better information than either system alone) may enable the real-time identification of structures within either visualization platform (e.g., laparoscopic or endoscopic).

An example of fiducial alignment is provided herein. The global coordinate system may be established based on a pre-operative CT image. A laparoscopic system and endoscopic system may have their own local coordinate systems. Through the use of multiple fiducial markers (e.g., seen by each system), a reference coordinate system (e.g., a reference configuration) may be defined relative to the global coordinate system (e.g., with deformations roughly accounted for based on the current image). Coordinating the fiducials in real time may provide greater clarity and/or accuracy in the local coordinate systems for each system. The object registration may enable interpretation of structures in the current image that the local technology (e.g., visual laparoscopic camera) cannot definitively identify.

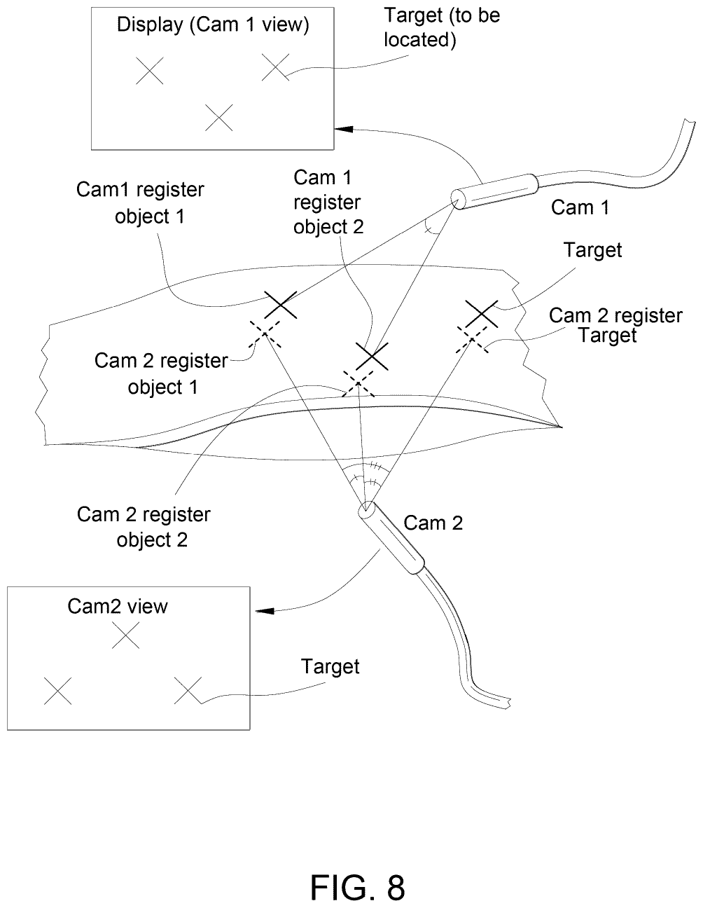

In an example, gallbladder stones may be occluded in the common bile duct. The common bile duct may be opened up and the gallbladder (e.g., which is the source of the stones) may be removed. This may involve multiple (e.g., two or three) cooperative smart systems (e.g., a robotic flexible endoscope, a robotic laparoscope, and optionally a high intensity ultra sound therapeutic system). At least two of the systems may interchange data, registrations, and/or the like. The systems may work from both sides of a tissue barrier (e.g., inside and outside an organ wall), as shown in FIG. 8. A third system may receive targeting information from one or more of the other systems. The third system may not provide data back to the other systems.

FIG. 8 illustrates an example of two scopes registering objects in their field of view. As shown, the scopes may be on opposite sides of a tissue barrier (e.g., a laparoscopic side and an endoscopic side). A first scope (labeled Cam1) may identify two objects (e.g., object 1 and object 2) in its field of view. The first scope may register the two objects. The first scope may determine an angle between the two objects.

As illustrated, a second scope (labeled Cam2) may also register the two objects. The second scope may further register a target object. The second scope may send location information (e.g., related to its field of view) to the first scope or to an independent system. The first scope or other system may determine, based on the first scope's registration of the two objects and the second scope's registration of the two objects and location information of the target object, where the target object is located in the first scope's field of view. For example, the first scope or other system may compare the first scope's registration of the two objects to the second scope's registration of the two objects to determine a coordinate transform between the views of the scopes. For example, in FIG. 8, the transform may indicate that the view of the first scope is a reflection of the second scope's view over the horizontal axis. The first scope may use the transform to determine where the target object is located in the first scope's field of view.

Gallstones may cause problems such as pain (e.g., biliary colic) and gallbladder infections (e.g., acute cholecystitis). Gallstones may migrate out of the gallbladder. The gallstones may become trapped in the tube between the gallbladder and the small bowel (e.g., common bile duct). In the common bile duct, the gallstones may obstruct the flow of bile from the liver and gallbladder into the small bowel. This may cause pain, jaundice (e.g., yellowish discoloration of the eyes, dark urine, and pale stools), and sometimes severe infections of the bile (e.g., cholangitis). Between 10% and 18% of people undergoing cholecystectomy for gallstones may have common bile duct stones.

The pancreas is a long, flat gland that sits tucked behind the stomach in the upper abdomen. The pancreas produces enzymes that help digestion and hormones that help regulate the way the body processes sugar (e.g., glucose). Pancreatitis refers to inflammation of the pancreas. Pancreatitis may occur if the bile duct is clogged and the pancreas enzymes cannot be transferred into the small intestines. In this case, the enzymes begin to break down the pancreas itself (e.g., from the inside out). Pancreatitis may occur as acute pancreatitis (e.g., pancreatitis that appears suddenly and lasts for days). Some people develop chronic pancreatitis (e.g., pancreatitis that occurs over many years). Mild cases of pancreatitis improve with treatment. Severe cases may cause life-threatening complications.

Treatment of gallstones may involve removal of the gallbladder and the gallstones from the common bile duct. There are several ways to remove the gallbladder and gallstones. Surgery may be performed to remove the gallbladder. The surgery may be performed through a large incision through the abdomen (e.g., open cholecystectomy). The surgery may be performed using keyhole techniques (e.g., laparoscopic surgery). Removal of the trapped gallstones in the common bile duct may be performed at the same time as the open or keyhole surgery. An endoscope (e.g., a narrow flexible tube equipped with a camera) may be inserted (e.g., through the mouth) into the small bowel to allow removal of the trapped gallstones from the common bile duct. This procedure may be performed before, during, and after the surgery to remove the gallbladder. A surgeon may have to determine whether to remove the common bile duct stones during surgery to remove the gallbladder (e.g., as a single-stage treatment), or as a separate treatment before or after surgery (e.g., a two-stage treatment).

The pre-operative CT or MRI may have a larger perspective view of the surgical area (e.g., compared to intraoperative imaging devices). The CT or MRI may come with limitations. For example, the CT or MRI may be taken at an earlier point of time and the anatomy and disease state progression may be different from that at the time of surgery. The pre-operative imaging (e.g., CT or MRI) may be taken in the supine portion, while surgery may be performed in the Trendelenburg position. This change in position may cause the organs and surgical site to distort (e.g., shift compared to the pre-operative imaging). The distortion may not be uniform with the retroperitoneal and peritoneal (e.g., structures behind the peritoneum or in front of the peritoneum, respectively) organs adjusting differently (e.g., due to different levels of fixation to more rigid portions of the body). For example, retroperitoneal structures may move less with changes in anatomic position (e.g., because such structures are more rigidly fixated to the back wall of the cavity).

In this example, the first surgical imaging device may be an intraoperative imaging device and the second surgical imaging device may be a pre-operative imaging device. The transformation between the first reference geometry and the second reference geometry may be calculated based on a difference between a first patient position during capture of the first imaging data stream and a second patient position during capture of the second imaging data stream, as described herein.

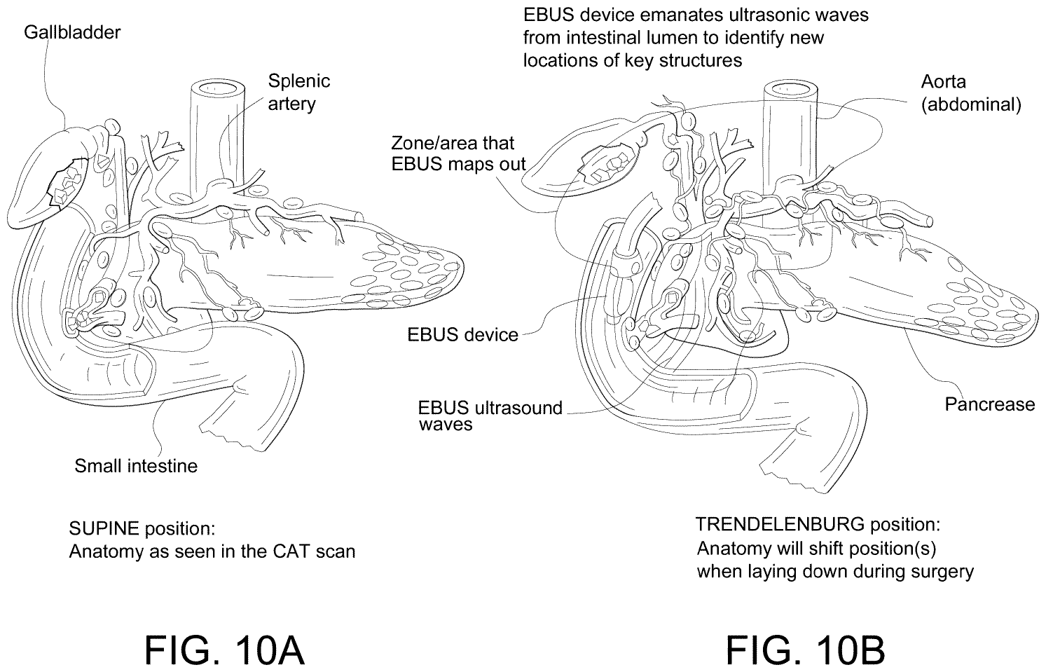

The change in position and its effect on the location of some anatomical structures is illustrated in FIGS. 10A and 10B. As shown, the gallbladder may be moved away from the small intestine and the pancreas may be elongated in Trendelenburg position. Similarly, gallstones may migrate downstream during the time between pre- and intra-operative imaging. GI motility or other movement of objects through the organs may distort the synchronization of imaging registration.

Patient anatomic positioning may be compensated for during surgery. The time dependent variable may be addressed (e.g., after compensating for the patient anatomic positioning). The pre-operative imaging may be used for guidance, registration, or identification of differing real-time surgery imaging. For example, a first surgical imaging device may be an intraoperative imaging device and a second surgical imaging device may be a pre-operative imaging device. The first imaging data stream may be captured with a patient in a first patient position and the second imaging data stream may be captured with the patient in a second patient position.

In this case, the anatomy may be adjusted to that of the surgical position. The adjustment may be done by identification of common landmarks that are less- or non-moving landmarks, 3D shape comparison, and/or local fiducial marker synchronization. For example, the reference landmark may be an anatomical structure that has relatively little movement between capture of the first and second imaging data streams. In the example illustrated in FIGS. 10A and 10B, the aorta and/or splenic artery may be used as the common landmark(s) since those structures moved very little, if at all, between the two images.

As illustrated, anatomy that is more controlled (e.g., with less movement and more constant 3D shape) may be used to differentiate structures. The system may determine local characteristics of the other structures to identify those structures. The system may display the adjusted images to the user. The adjusted images may be used for annotation and/or later navigation & intervention.

Local ultrasound imaging (e.g., using an EBUS ultrasound sensor) may identify sub-surface anatomical structures and/or foreign objects (e.g., within adjacent hollow structures like the common bile duct). A surgeon may use an EBUS ultrasound and/or visual light imaging flexible endoscope. The sensor may be enclosed in a balloon filled with saline (e.g., which makes contacting the duodenum wall easier for ultrasound imaging). The system may differentiate the objects from one another. The system may mark the objects' registration (e.g., for later interaction). Ultrasound may provide a good size and shape interpretation of the objects. Ultrasound may not provide information regarding other features (e.g., object density or composition).

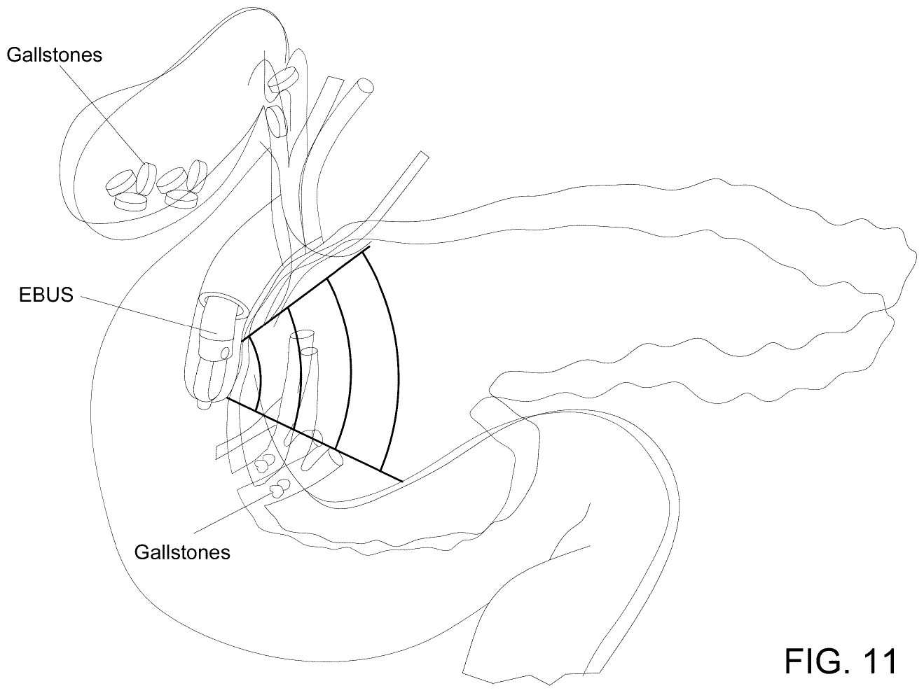

The system may differentiate between detected objects (e.g., two detected objects in close proximity with similar size and shape). For example, the system may utilize the adjusted pre-operative imaging map (e.g., with focus on the local area of concern). This may account for retroperitoneal and peritoneal positioning to forecast where structures in pre-operative images are after the patient is moved. FIG. 11 illustrates an example display in which pre-operative and intra-operative imaging are overlaid on one another. The overlaid images may enable the surgeon to identify structures in intra-operative images based on their relative location/shape compared to the pre-operative images.

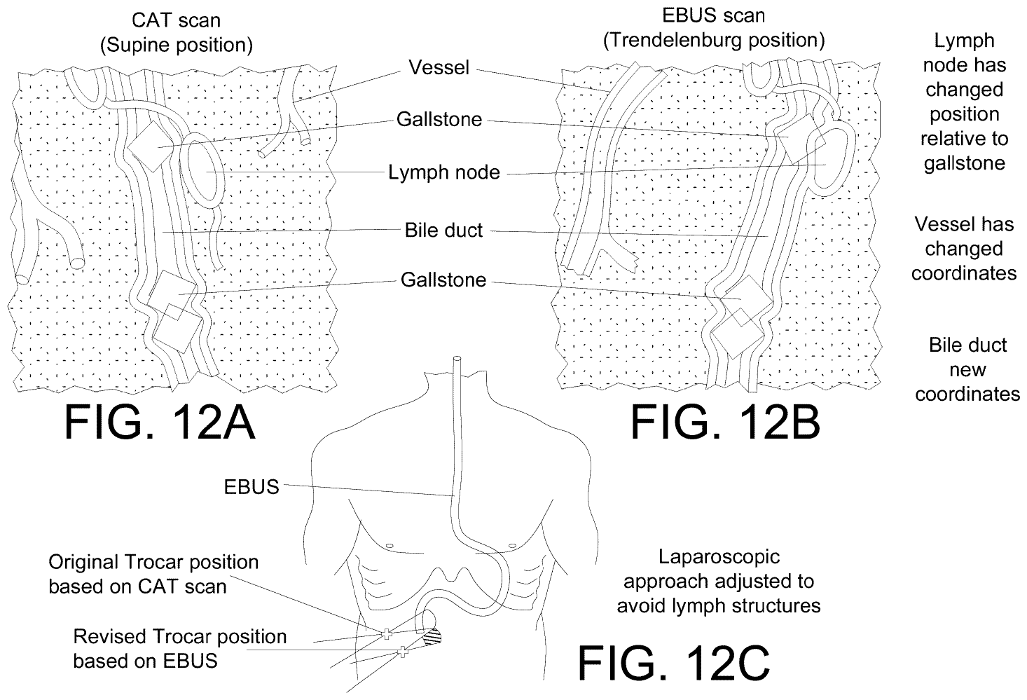

The system may utilize mapping of adjacent or enclosed secondary structures. For example, a gallstone may be in the hollow adjacent bile duct and the lymph nodes may not be in that bile duct. If the common bile duct location can be imaged or predicted (e.g., like a street mapping program), the system may be able to differentiate objects that are moving within the common bile duct. FIGS. 12A-C illustrate the change in location of anatomical structures (e.g., gallstones and lymph nodes) between pre- and intra-operative imaging.

If the system were unable to adequately identify and annotate the structures, a third (e.g., gray) visualization may be used to denote a structure having no identifier. The surgeon may move the scope closer to the unidentified object. The surgeon may identify the object (e.g., visually using the visual light imaging scope on the flexible endoscope).

Lymph nodes may not be part of the cholecystectomy (gallbladder removal) or Endoscopic Retrograde Cholangiopancreatography (ERCP) stone removal from the bile duct. The gallstones may be addressed/removed during the procedure. As the structures are identified, the annotation of the structures may allow the user to approach the structure (e.g., for removal). The structures may be noted for other vision system to use for common registration. The laparoscope may be able to differentiate gallstones in the duct from lymph nodes. The location of the gallstones may help the system determine where to transect (e.g., so as to not leave any remaining stone in the duct that may cause later complications).

Situational awareness may be applied to help determine differences between structures (e.g., that may not be definitively identified otherwise). For example, a gallstone and a lymph node may appear similar (e.g., similar size, density, etc.). With knowledge of the anatomy (e.g., such as the biliopancreatic duct structures), the surgeon may know that objects residing within the duct are gallstones and not a lymph node. Similarly, if the object resides outside the duct, the object cannot be a gallstone.

Pre-operative images may be adjusted to fit real time imaging. Once the local coordinate systems are defined (e.g., the local coordinate systems may be updated real time), an analysis of the available images (e.g., from multiple sources) may be performed to identify structures (e.g., key structures of interest). CT may be able to show structural elements and landmarks (e.g., the common bile duct, biliopancreatic duct, ampulla of Vater, gallbladder, liver, pancreas, duodenum, and/or gallstones, although results may be inconclusive). With a local coordinate system known relative to an adjusted global coordinate system, information from EBUS may identify gallstones, ampulla of Vater, common bile duct, biliopancreatic duct, etc. This information may be used to confirm the identity of structures from other views. For example, with coordination, the laparoscopic view may identify structures for the surgeon (e.g., based on the information gathered and interpreted from the CT image and/or the ultrasound image). This approach may be used to identify lymph nodes and/or other structures of interest.

Differentiating gallstones from other structures may be difficult on the full body CT. Differentiating gallstones from other structures may be easier under local ultrasound. The system may re-establish registration (e.g., compensate for patient positioning changes). The system may make a more decisive determination as to whether a structure is a gallstone. The system may tag the gallstones for communication to other smart system(s). The other system(s) may use the in-surgery registration to obtain location and positioning information. The presence of kidney stones and gallstones via CT may be detected 95-98% of the time.

The accuracy of a low dose CT to discriminate a gallstone from adjacent normal anatomic structures may be lower than that of other imaging devices. Radiologists may only be able to discriminate all the stones and there locations 72-100% of the time. Low dose CT scans may correctly identify kidney stones 90%-98% of the time. Low dose CT scans may correctly confirm no kidney stones were present 88%-100% of the time. Ultra-low dose CT scan may correctly identify kidney stones 72%-99% of the time. Ultra-low dose CT scans may correctly confirm no kidney stones were present 86%-100% of the time.

External ultrasonic imaging may be as accurate as CT (e.g., and possibly less accurate) in the identification of stones. Local ultrasound (e.g., on a flexible endoscope) may be more (e.g., significantly more) accurate in identifying stones and determining the location of stones. The local system may be used to determine the in-surgery registrations, determine whether a structure is a stone or anatomy, and/or adjusting other imaging systems to match the imaging seen in real time.

The EBUS scope may be removed (e.g., after the imaging, registration and annotation are complete). The EBUS may be replaced with a standard visualization scope. The EBUS system may have (e.g., may only have) a 2.2 millimeter working channel. A basket snare retrieval tool may use a 2.8 mm working channel to operate and retrieve stones in the common bile duct. A guide wire may be introduced down the working channel of the EBUS device. The EBUS scope may be removed while leaving the guide wire in place. A larger working channel visual scope may be introduced over the wire to get it back in the same position. The registration and imaging may be used to guide the new scope into the proper location for bile duct introduction and stone retrieval.

Some of the stones to be retrieved may be too far up the duct (e.g., and therefore out of reach of the physical retrieval basket. In this case, the doctor may use an extracorporeal (e.g., from outside the body) Shock Wave Lithotripsy. The Shock Wave Lithotripsy may generate sound waves that are focused on the stone from an outside high intensity ultrasound generator. The sound waves may crush the stone. The endoscopy visualization and pre-registrations may be used to target the stone. The sound waves may be used to crush the stone into small pieces.

Once the stones are handled (e.g., retrieved or crushed) the laparoscopic-side surgery may begin. The surgeon may select where in the duct from the gallbladder to transect. The duct, the vein, and the artery feeding the gallbladder may be transected with an endocutter. The gallbladder may then be removed. A stone may be located near the gallbladder (e.g., within the duct). To remove the stone (and the stones in the gallbladder), the transection may be made below the stone location. In this case, the stone may remain in the extracted specimen side of the transection (e.g., not the remnant side). To do this, the imaging of the EBUS system and the registration of the stone in the duct may be used to determine the exact transection site of the duct.

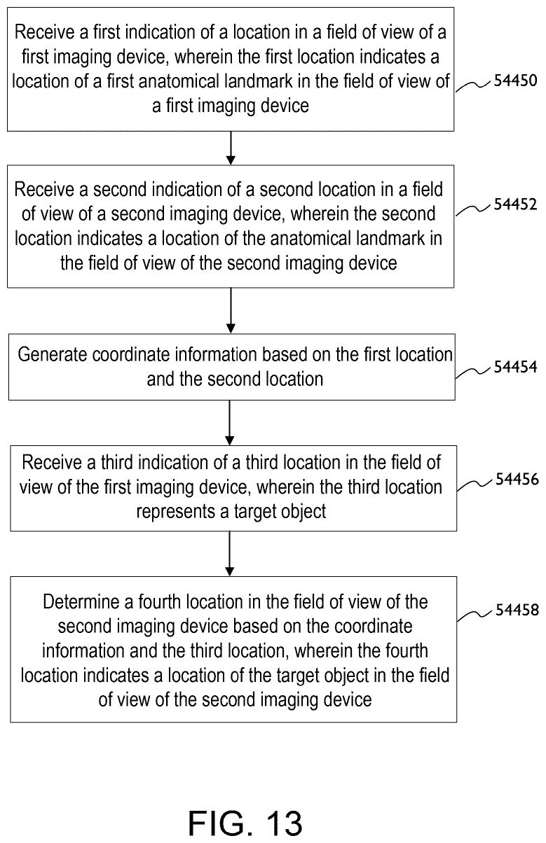

FIG. 13 illustrates an example method that may be performed by a surgical instrument. As shown at 54450, the method may involve capturing a first imaging data stream of a visualized surgical structure. The method may involve, at 54452, communicating registration information with a second surgical imaging device capturing a second imaging data stream of the visualized surgical structure. The method may involve, at 54454, identifying, based on the registration information, a first location, within the first imaging data stream, associated with the visualized surgical structure. The first location may be determinable based on: a first reference geometry associated with a field of view of the first surgical imaging device, a second reference geometry associated with a field of view of the second surgical imaging device, a transformation between the first reference geometry and the second reference geometry, and a reference landmark common to the first imaging data stream and the second imaging data stream.

3Di GmbH may use CT scans to create precisely adapted patient-specific implants. The CT scans may be taken of the patient. The data may be transferred to 3Di, which will process the data and create a virtual patient model. Once the patient model is confirmed, 3Di will create a 3D implant model that may be reviewed/confirmed by a doctor. The implant model may then be manufactured and sent to site for implant.

In this example, multiple scan sources may be combined to further enhance the model created (e.g., instead of only utilizing a CT scan). For example, in addition to CT imaging, ultrasound and/or MRI may be used to develop the 3D model.

The 3Di processor may interact with the CT scanning system (e.g., to identify inputs that do not generate correctly and/or anomalies identified during the virtual model). This coordination may minimize potential errors or differences within a patient to create a more accurate representation of the implant.

Photogrammetry may be used to enable one or more feature(s) described herein. Photogrammetry may use photographic cameras to obtain information about the 3D world. Photogrammetric measurements may be performed by recording a light ray in a photographic image. The light ray may correspond to observing a direction from the camera to the 3D scene point where the light was reflected or emitted. From this relation, the cameras may be oriented relative to each other, or relative to a 3D object coordinate frame, to reconstruct unknown 3D objects through triangulation.

For single-view geometry, a collinearity equation may be used. The mapping with an ideal perspective camera may be decomposed into two steps: a transformation from object coordinates to camera coordinates (e.g., sometimes referred to as exterior orientation), and a projection from camera coordinates to image coordinates using the cameras' interior orientation. The exterior orientation may be achieved by a translation from the object coordinate origin to the origin of the camera coordinate system (e.g., the projection center) and a rotation that aligns the axes of the two coordinate systems.

With the Euclidean object coordinates X₀^{e}=[X₀, Y₀, Z₀]^{T} of the projection center and the 3×3 rotation matrix R, the relationship may be expressed as:

$$\ddot{x} = MX = [ \begin{matrix}R & 0 \\ {0}^{⊤} & 1\end{matrix} ] [ \begin{matrix}1 & - {X}_{0}^{e} \\ {0}^{⊤} & 1\end{matrix} ] X$$

where upper case letters (e.g., X) refer to object coordinates, lower case letters with a tilde (e.g., \tilde{x}) to camera coordinates, and plain lowercase letters (e.g., x) to image coordinates.

With respect to the camera coordinate system, the image plane may be perpendicular to the z-axis. The z-axis may be referred to as the principal ray. The z-axis may intersect the image plane in the principal point, which has the camera coordinates \tilde{x}_{H}=\tilde{t}·[0, 0, c, 1]^{T} and the image coordinates x=t·[x_{H}, y_{H}, 1]^{T}. The distance c between the projection center and the image plane is the focal length (or camera constant). The perspective mapping from camera coordinates to image coordinates may be written as:

$$x = [ K \vert 0 ] \tilde{x} = [ \begin{matrix}c & 0 & {x}_{H} & 0 \\ 0 & c & {y}_{H} & 0 \\ 0 & 0 & 1 & 0\end{matrix} ] \tilde{x}$$

This relation holds if the image coordinate system has no shear (e.g., orthogonal axes, respectively pixel raster) and isotropic scale (e.g., same unit along both axes, respectively square pixels). If a shear s and a scale difference m are present, they amount to an affine distortion of the image coordinate system, and the camera matrix becomes:

$$K = [ \begin{matrix}c & cs & {x}_{H} \\ 0 & c ( 1 + m ) & {y}_{H} \\ 0 & 0 & 1\end{matrix} ]$$

with five parameters for the interior orientation.

By concatenating the two steps from object to image coordinates, the final projection (e.g., the algebraic formulation of the collinearity constraint) may be expressed as:

$$x = λ PX ∝ PX = KR [ I \vert - {X}_{0}^{e} ] X$$

If an object point X and its image x are both given at an arbitrary projective scale, they will only satisfy the relation up to a constant factor. To verify the constraint, (e.g., check whether x is the projection of X, one can use the relation:

$$x \times PX = {[ x ]}_{x} PX = 0$$

Note that due to the projective formulation only two of the three rows of this equation are linearly independent. Given a projection matrix P, the interior and exterior orientation parameters may be extracted. For example, the following equation may be used to determine the parameters:

$$P = [ M \vert m ] = [ KR \vert - {KRX}_{0}^{e} ]$$

The translation part of the exterior orientation immediately follows from X₀^{e}=−M⁻¹ m. The rotation may (e.g., by definition) be an orthonormal matrix. The calibration may (e.g., by definition) be an upper triangular matrix. Both properties may be preserved by matrix inversion. The two matrices may be found by QR decomposition of M⁻¹=R^{T}K⁻¹ (or by RQ decomposition of M).

A person of ordinary skill in the art will appreciate that other geometries (e.g., two-view geometry) may be used to enable the features described herein.

What is claimed:

1. A first surgical imaging device comprising:
a processor configured to:
capture a first imaging data stream of a visualized surgical structure;
communicate registration information with a second surgical imaging device capturing a second imaging data stream of the visualized surgical structure; and
identify, based on the registration information, a first location, within the first imaging data stream, associated with the visualized surgical structure, wherein the first location is determinable based on: a first reference geometry associated with a field of view of the first surgical imaging device, a second reference geometry associated with a field of view of the second surgical imaging device, a transformation between the first reference geometry and the second reference geometry, and a reference landmark common to the first imaging data stream and the second imaging data stream.

2. The first surgical imaging device of claim 1, wherein the processor is further configured to calculate the transformation between the first reference geometry and the second reference geometry based on a first physical orientation of the first surgical imaging device, a second physical orientation of the second surgical imaging device, and the reference landmark.

3. The first surgical imaging device of claim 1, wherein the first surgical imaging device is an intraoperative imaging device and the second surgical imaging device is a pre-operative imaging device, and wherein the processor is further configured to calculate the transformation between the first reference geometry and the second reference geometry based on a difference between a first patient position during capture of the first imaging data stream and a second patient position during capture of the second imaging data stream.

4. The first surgical imaging device of claim 1, wherein the first surgical imaging device is an intraoperative imaging device and the second surgical imaging device is a pre-operative imaging device, wherein the first imaging data stream is captured with a patient in a first patient position and the second imaging data stream is captured with the patient in a second patient position, and wherein the reference landmark comprises an anatomical structure that has relatively little movement between capture of the first and second imaging data streams.

5. The first surgical imaging device of claim 1, wherein the visualized surgical structure comprises an object to be removed from a patient, and wherein the reference landmark comprises an anatomical structure of the patient.

6. The first surgical imaging device of claim 1, wherein the first surgical imaging device is an endoscopic device, the second surgical imaging device is a laparoscopic device, the first surgical imaging device and the second surgical imaging device are separated by a tissue barrier.

7. The first surgical imaging device of claim 6, wherein the processor is further configured to calculate the transformation between the first reference geometry and the second reference geometry based on one or more lights projected, by the laparoscopic device, through the tissue barrier, and detected by the endoscopic device.

8. The first surgical imaging device of claim 1, wherein the processor is further configured to:
generate an augmented reality (AR) overlay of the first imaging data stream, the second imaging data stream, based on the transformation between the first reference geometry and the second reference geometry, wherein the AR overlay depicts the first imaging data stream and the second imaging data stream overlapped onto one another based on the transformation; and
output the AR overlay to a display device for displaying.

9. The first surgical imaging device of claim 1, wherein the first surgical imaging device and the second surgical imaging device are capturing video data at the same time.

10. A method performed by a first surgical imaging device, the method comprising:
capturing a first imaging data stream of a visualized surgical structure;
communicating registration information with a second surgical imaging device capturing a second imaging data stream of the visualized surgical structure; and
identifying, based on the registration information, a first location, within the first imaging data stream, associated with the visualized surgical structure, wherein the first location is determinable based on: a first reference geometry associated with a field of view of the first surgical imaging device, a second reference geometry associated with a field of view of the second surgical imaging device, a transformation between the first reference geometry and the second reference geometry, and a reference landmark common to the first imaging data stream and the second imaging data stream.

11. The method of claim 10, wherein the method further comprises calculating the transformation between the first reference geometry and the second reference geometry based on a first physical orientation of the first surgical imaging device, a second physical orientation of the second surgical imaging device, and the reference landmark.

12. The method of claim 10, wherein the first surgical imaging device is an intraoperative imaging device and the second surgical imaging device is a pre-operative imaging device, and wherein the method further comprises calculating the transformation between the first reference geometry and the second reference geometry based on a difference between a first patient position during capture of the first imaging data stream and a second patient position during capture of the second imaging data stream.

13. The method of claim 10, wherein the first surgical imaging device is an intraoperative imaging device and the second surgical imaging device is a pre-operative imaging device, wherein the first imaging data stream is captured with a patient in a first patient position and the second imaging data stream is captured with the patient in a second patient position, and wherein the reference landmark comprises an anatomical structure that has relatively little movement between capture of the first and second imaging data streams.

14. The method of claim 10, wherein the visualized surgical structure comprises an object to be removed from a patient, and wherein the reference landmark comprises an anatomical structure of the patient.

15. The method of claim 10, wherein the first surgical imaging device is an endoscopic device, the second surgical imaging device is a laparoscopic device, the first surgical imaging device and the second surgical imaging device are separated by a tissue barrier.

16. The method of claim 15, wherein the method further comprises calculating the transformation between the first reference geometry and the second reference geometry based on one or more lights projected, by the laparoscopic device, through the tissue barrier, and detected by the endoscopic device.

17. The method of claim 10, wherein the method further comprises:
generating an augmented reality (AR) overlay of the first imaging data stream, the second imaging data stream, based on the transformation between the first reference geometry and the second reference geometry, wherein the AR overlay depicts the first imaging data stream and the second imaging data stream overlapped onto one another based on the transformation; and
outputting the AR overlay to a display device for displaying.

18. The method of claim 10, wherein the first surgical imaging device and the second surgical imaging device are capturing video data at the same time.
